(19) United States
(12) Patent Application Publication
(10) Pub. No.: US 2008/0275424 A1
(43) Pub. Date: Nov. 6, 2008

(54) METHODS AND DEVICES FOR TREATMENT OF OBESITY

(76) Inventors: Rajiv Doshi, Stanford, CA (US); David Miller, Cupertino, CA (US); William N. Aldrich, Napa, CA (US)

Correspondence Address: ORRICK, HERRINGTON & SUTCLIFFE LLP, SUITE 1600, FOUR PARK PLAZA, IRVINE, CA, 92614-2558, US

(21) Appl. No.: 11/743,859

(22) Filed: May 3, 2007

(51) Int. Cl. A61N 7/00

(52) U.S. Cl. 604/506; 128/898; 601/2; 607/40; 607/89

(57) ABSTRACT

The present invention provides methods and systems for treating obesity by creating an interruption of the vagal nerve, preferably in the region of the esophagus, by minimally or noninvasive means.

### FIELD OF THE INVENTION

The field of the present invention is methods and devices for treating obesity, and more particular, methods and devices for treating obesity by disrupting the vagal nerve.

### BACKGROUND INFORMATION

Obesity has become an ever-increasing health problem. While such voluntary weight reduction programs as dieting and exercise have been helpful for some, many obese persons have required surgery to address their obesity problem. Two such surgical procedures are vertical banded gastroplasty (VBG) and the Roux-en-Y gastric bypass procedure. Both such procedures are now well known, but they are invasive in nature and involve reducing the size of the stomach. While this procedures have demonstrated a reasonable level of efficacy, there is a need for an improvement in the treatment of obesity that would avoid invasive surgery and providing effective treatment of obesity.

### SUMMARY

The present invention provides methods and systems for treating obesity by creating an interruption of the vagal nerve by minimally or noninvasive means. While the present invention is not to be tied to any particular theory of operation, it appears that a hunger signal is expressed by ghrelin, a peptide primarily produced in the stomach, and transmitted to the brain through the vagal nerve. The literature e.g., “The Role of the Gastric Afferent Vagal Nerve in Ghrelin-Induced Feeding and Growth Hormone Secretion in Rats,” Gastroenterology 2002:123:1120-1128 (October 2002) by Yukari Date et al. and “Gastroplasty for Obesity: Long-term Weight Loss Improved by Vagotomy,” World Journal of Surgery, Vol. 17, No. 1, January/February 1993, by Kral et al., supports this theory. The Date et al. article concluded that blockade of the gastric vagal afferent abolished ghrelin-induced feeding in rats and the Kral et al. article concluded that vagotomy combined with gastroplasty was more effective in controlling weight loss than gastroplasty alone. These articles are incorporated by reference herein.

In an embodiment, a flexible endoscope is advanced down the esophagus to an area of interest within the esophagus or stomach. The endoscope may be guided to and positioned at the area of interest by the endoscopic viewer or other means. A needle is then advanced through the working channel of the endoscope and inserted into the esophageal or gastric wall to position the needle tip near the vagal nerve to be disrupted. A chemical agent is then delivered from the needle to bathe the vagal nerve in the agent (or to deposit or expose the tissues surrounding the vagal nerve to the agent), thereby disrupting the nerve. The agent may be any agent that permanently or temporarily interferes with the transmission of signals via the vagal nerve, including but not limited to toxins causing cellular death or damage, and agents blocking the transmission of signals via the vagal nerve. Other suitable agents include hot or cold liquids, gels or other fluids having sufficient temperature to cause cellular death or damage. In an embodiment, the agent is injected within a potential space surrounding the vagal nerve that is defined on one side by the esophagus and on the other side by the phreno-esophageal ligaments. Injection or deposition of the agent into this potential space allows the agent to dwell in the space surrounding the vagal nerve, thereby increasing exposure of the vagal nerve to the agent. In another embodiment, the agent is injected into fat around or near the vagal nerve wherein the fat serves as a deposit within which the agent can dwell and/or from which the agent can elute to increase exposure of the vagal nerve to the agent. After a predetermined time, the agent may be suctioned away from the vagal nerve to control exposure to the agent.

In another embodiment, a flexible endoscope is advanced down the esophagus to a position near an interior wall of the esophagus or stomach. A needle or other cutting instrument is then advanced through the working channel of the endoscope (or may be located on or attached to the outside of the endoscope) and used to pierce a small opening through the esophageal or gastric wall. The distal portion of the endoscope is then advanced through the small opening into the abdominal or thoracic cavity. In the abdominal or thoracic cavity, the distal portion of the endoscope is retroflexed, i.e., bent upwards, to provide medical devices with access to vagal nerves or branches on the outer surface of the esophagus or stomach or other organs. This is done by advancing the medical device to the desired vagal nerve through the working channel of the endoscope. In an embodiment, the medical device thermally disrupts the desired vagal nerve by delivering a hot or cold liquid on the vagal nerve. In another embodiment, the medical device disrupts the vagal nerve by applying radio frequency (RF), microwave, high intensity ultrasound, and/or laser energy to the vagal nerve. In another embodiment, the medical device mechanically disrupts the vagal nerve by dissecting the vagal nerve with a cutting instrument, e.g., scissors, and/or ligating the vagal nerve. In yet another embodiment, the medical device deploys a circular or other band to mimic the effects of a laparoscopic band. The vagal nerve may be disrupted using any one of or a combination of the above methods.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims. It is also intended that the invention not be limited to the details of the example embodiments.

### BRIEF DESCRIPTION OF THE FIGURES

FIG. 1 is a diagrammatic illustration of the general anatomy of the stomach and esophagus.

FIG. 2 illustrates a method for performing a vagotomy by injecting an agent into a space surrounding the vagal nerve according to an embodiment of the present invention.

FIG. 3 illustrates a flexible endoscope installed in the abdominal cavity through a small opening in the stomach according to an embodiment of the present invention.

FIG. 4 illustrates a flexible endoscope installed in the thoracic cavity through a small opening in the esophagus according to an embodiment of the present invention.

FIG. 5 illustrates a medical device advanced through the working channel of the endoscope to thermally disrupt a vagal nerve on the outer surface of the esophagus or stomach according to an embodiment of the present invention.

FIG. 6 illustrates a medical device advanced through the working channel of the endoscope to mechanically disrupt a vagal nerve on the outer surface of the esophagus or stomach according to an embodiment of the present inventions.

FIG. 7 illustrates a medical device advanced through the working channel of the endoscope to deploy a circular or other band to mimic the effects of a laparoscopic band.

### DETAILED DESCRIPTION

Before turning to the manner in which the present invention functions, it is believed that it will be useful to briefly review the anatomy of the stomach and the esophagus. The esophagus is a muscular tube that carries food from the throat to the stomach and which passes through the diaphragm. The top end of the esophagus is the narrowest part of the entire digestive system and is encircled by a sphincter (circular muscle) that is normally closed but can open to allow the passage of food. There is a similar sphincter at the point where the esophagus enters the stomach. The walls of the esophagus consist of strong muscle fibers arranged in bundles, some circular and others longitudinal. The inner lining of the esophagus consists of smooth squamous epithelium (flattened cells).

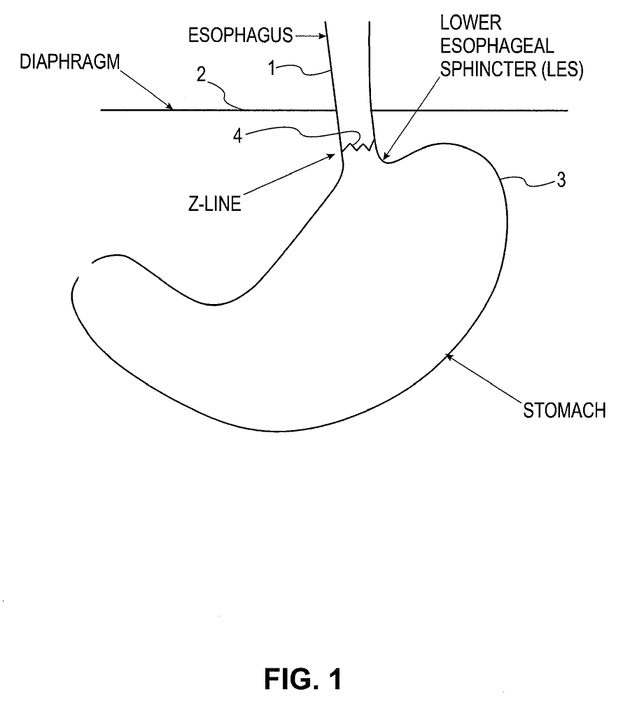

As shown in FIG. 1, the esophagus 1 extends through the diaphragm 2 into the stomach 3. Vagal nerve branches (not shown) extend from the stomach along the outer wall of the esophagus to the brain. The anatomy of the vagal nerve complex varies somewhat from person to person, but, common to all is a structure comprising multiple vagal nerve branches located on the outer wall of the esophagus which run generally longitudinally along the esophagus wall. Typically, there are two main branches, or trunks, of the vagal nerve which are located approximately 180° from each other on the outer wall of the esophagus.

To treat obesity, the present invention provides methods and devices for interrupting the vagal nerve, e.g., in the region of the esophagus. Depending on patient needs, it may be sufficient to interrupt only a portion of the fibers in the nerve. In this regard, it is to be noted that, in general, myelinated vagal nerve fibers, i.e., fibers that have an outer coating, are efferent. In contrast, afferent vagal nerves are unmyelinated and have no outer covering. For some patients, it may be sufficient to interrupt the function of only the afferent vagal fibers.

An exemplary flexible endoscope suitable for use in various methods of the present invention will now be discussed. The endoscope comprises a steerable, flexible tube adapted to pass through the esophagus and other cavities of the body. The endoscope may comprise a lighted endoscopic viewer for providing the operator with visualization from the distal end of the endoscope. The endoscopic viewer may include a lens at the distal end of the endoscope coupled to an external eyepiece and/or a video camera via an optical fiber. To illuminate the operative field, light may be provided at the distal end of the endoscope via an optical fiber connected to a light source. The flexible tube includes one or more working channels for advancing medical instruments through the endoscope and into the interior of the body from an opening at the distal end of the endoscope. This endoscope is intended to be exemplary only, as any endoscope capable of performing the functions of the inventions may be used.

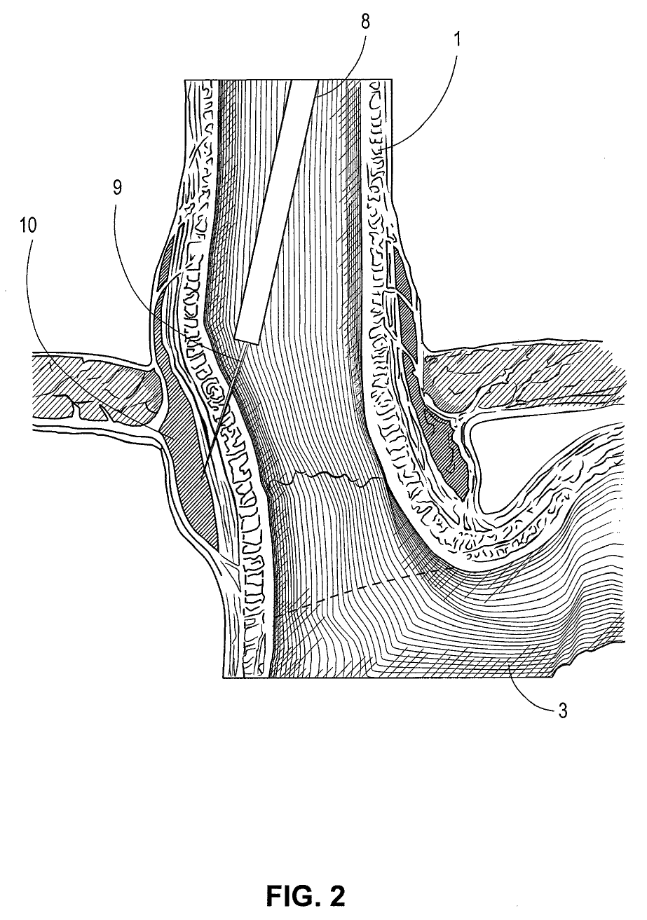

FIG. 2 illustrates a method for performing a vagotomy according to an embodiment. In this embodiment, a flexible endoscope 8 is inserted into the esophagus 1 through the mouth, and advanced down the esophagus 1 to an area of interest within the esophagus 1 or stomach 3. The endoscope 8 may be guided to and positioned at the area of interest using the endoscopic viewer and/or other means. A needle 9 is then advanced through the working channel of the endoscope 8 and extended passed the distal end of the endoscope 8, as shown in FIG. 2. The needle 9 is inserted into the esophageal or gastric wall to position the needle tip near the vagal nerve to be disrupted. At this point, a chemical agent is delivered from the needle 9 to bathe the vagal nerve with the agent, thereby disrupting the nerve. Examples of suitable agents are given below. In some cases, the agent may be suctioned from the site of interest after a predetermined time (e.g., after 30 seconds or 5 minutes) through the needle or some other means. After administration of the agent, the needle 9 is withdrawn from the site. The needle may be inserted into another portion of the esophageal or gastric wall to treat another vagal nerve or branch. After treating all of the nerves of interest, the needle 9 is withdrawn from the working channel of the endoscope 8.

The locations for the injection(s) can be anywhere within the digestive track, but preferably at or near the level of the diaphragm. In an embodiment, the injection site is within a potential space 10 surrounding the vagal nerve that is defined on one side by the esophagus and on the other side by the phreno-esophageal ligaments, as shown in FIG. 2. Injection of the agent into this potential space 10 allows the agent to dwell in the space surrounding the vagal nerve, thereby increasing the amount of exposure of the vagal nerve to the agent. Because the potential space 10 is contained, the agent is prevented from damaging surrounding tissue, such as the heart, lungs, blood vessels, diaphragm, etc. Other suitable injection sites include fat around a vagal nerve. The fat serves as a deposit within which the agent can dwell to increase the amount of exposure of the vagal nerve to the agent. A hot or cold liquid can also be injected into the injection site to disrupt the vagal nerve, wherein the liquid is of sufficient temperature to damage the vagal nerve. For example, hot water may serve as the hot liquid.

The purpose of the chemical agent or other agents is to permanently or temporarily interfere with the transmission of signals that are normally sent via the vagal nerve. Interference of transmission can involve the induction of cell death of the neuron or transection or lysis of the axon or other part of the cell. Chemical agents that may cause cellular death or damage include ethanol, saline, or other noxious agents. Alternatively, chemical agents may be used to block transmission of signals via the vagal nerve. This may involve the application of a chemical agent to the axon or to the receptors on or near the nerve terminal or other parts of the cell. Representative agents include capsaicin, resiniferatoxin, atropine, botulinum toxin, or other neurotoxins. Neurotoxin is meant to cover any chemical which leads to the damage, lysis or death of a cell, or any chemical which disrupts the transmission of a signal of a cell. Suitable neurotoxins include those that inhibit release, uptake, or binding of various neurotransmitters, including acetylcholine, oxide, glycine, GABA, serotonin, dopamine, epinephrine, noresinephrine, etc. Representative neurotoxins include botulinum toxin, tetanus toxin, tetrodotix, bungotoxin, terodotoxin, conotoxin, and derivatives thereof, etc.

An alternative means of using neurotoxins is the instillation of neurotoxin into the lumen of a digestive system organ (including but not limited to the stomach, gall bladder, small intestines, etc.), with the goal of reducing or eliminating the ability of the organ to contract, peristalse, or sense various stimuli from within the lumen. Capsaicin, resiniferatoxin, botulinum toxin may be of particular interest, although other neurotoxins may be used depending on the desired effect and length of action.

The dosage of the agent that is administered to the patient is one that is effective to achieve the desired treatment outcome. The amount of any particular agent varies depending on the nature of the particular agent. The dose can be given as a single dose, or as divided doses over a span of hours, days, weeks, or months. The chemical agent may be administered to the patient in a pharmaceutical preparation that includes the chemical agent in a pharmaceutically acceptable agent. The active agent can be presented as a sterile pyrogen-free aqueous solution or dispersion or as a sterile powder for reconstitution into a sterile solution or dispersion.

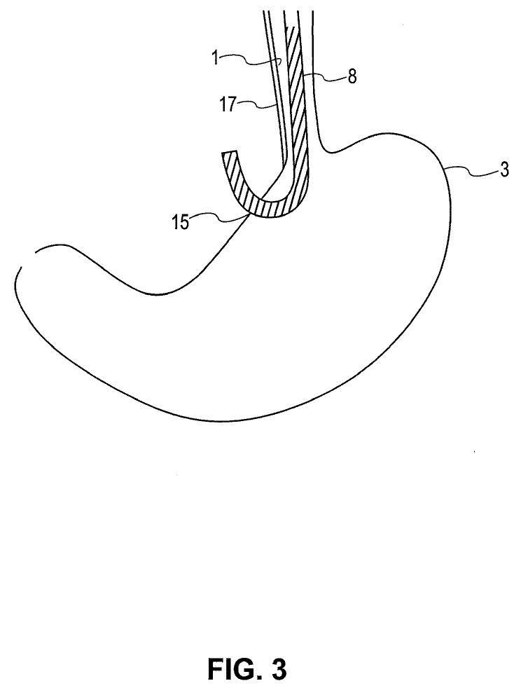

FIG. 3 illustrates in a diagrammatic manner installation of an endoscope 8 to perform a transgastric vagotomy according to an embodiment. In this embodiment, a flexible endoscope 8 is inserted into the esophagus 1 through the mouth, and advanced down the esophagus 1 into the stomach 3. The operator may use the endoscopic viewer or other means to guide the endoscope 8 within the body. The distal end of the endoscope 8 is then positioned near an interior wall of the stomach 3. A needle or other cutting instrument (not shown) is advanced through the working channel of the endoscope 8 and extended from the distal end of the endoscope 8 through the distal opening. The needle is used to pierce through the gastric wall to create a small opening or incision 15 through which the endoscope 8 can be advanced into the abdominal cavity. After the small opening 15 is created, the needle is withdrawn and the distal portion of the endoscope 8 is advanced through the opening 15 into the abdominal cavity. Once in the abdominal cavity, the distal portion of the endoscope 8 is retroflexed, i.e., bent upwards, as shown in FIG. 3. Endoscopes capable of being retroflexed are known in the art and are commercially available. In this configuration, the endoscope 8 provides medical device with access to vagal nerves or branches 17 on the outer surface of the esophagus 1 or stomach 3 through the working channel of the endoscope 8. This is done by advancing the medical device through the working channel into the abdominal or thoracic cavity to the desired vagal nerve. Methods for performing a vagotomy using the endoscope 8 in this configuration are given below.

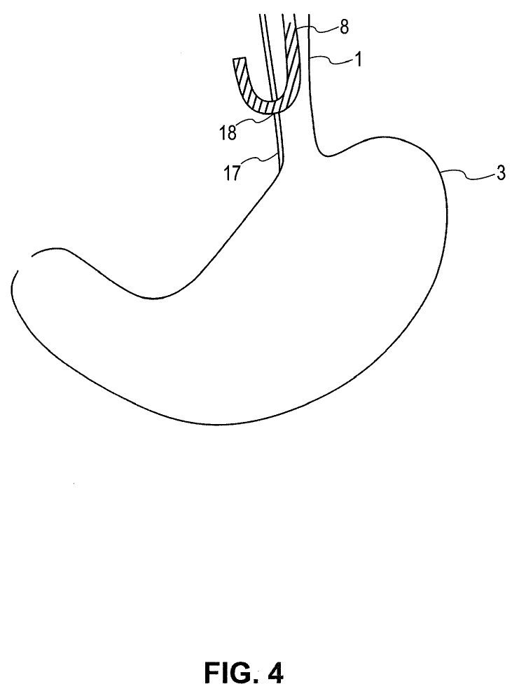

FIG. 4 illustrates another installation of an endoscope 8 to perform a vagotomy. In this embodiment, the endoscope 8 pierces through the esophageal wall instead of the gastric wall, and the distal portion of the endoscope 8 is advanced through the esophagus 1 into the thoracic cavity or abdominal cavity in an inferior or superior direction. In some instances, the distal portion of the endoscope 8 may be retroflexed in the thoracic cavity to provide medical devices with access to vagal nerves or branches 17 on the outer surface of the esophagus 1 through the working channel of the endoscope 8.

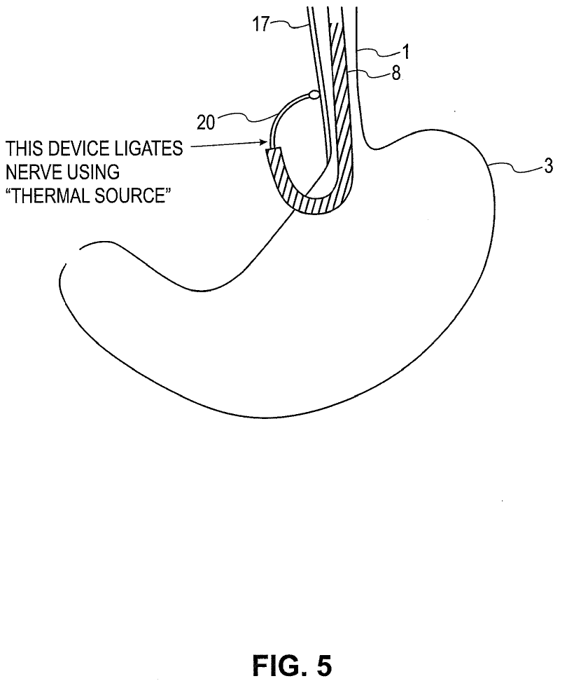

FIG. 5 illustrates an embodiment, in which a vagotomy is performed by thermally disrupting on one or more vagal nerves 17 on the outer surface of the esophagus 1 or stomach 3. In this embodiment, one or more medical devices 20 are advanced into the abdominal or thoracic cavity through the working channel of the endoscope 8. Although the medical device 20 is shown entering the abdominal cavity through the endoscope 8, the medical device 20 can also enter the thoracic cavity, e.g., through the endoscope 8 in FIG. 4, to perform the vagotomy. The medical device 20 may be visually guided to and positioned near the desired vagal nerve using endoscopic viewer and/or other means, e.g., X-ray. To thermally disrupt the desired vagal nerve 17, the medical device 20 may deliver a hot or cold liquid on the desired vagal nerve. To do this, the medical device 20 may comprise a needle or delivery port coupled to a liquid source. The medical device 20 may also disrupt the desired vagal nerve by applying radio frequency (RF), microwave, ultrasound (e.g., high intensity ultrasound), and/or laser energy to the vagal nerve 17. To do this, the medical device 20 may comprise an electrode, ablation needle, and/or ultrasound transducer at its distal tip. The medical device 20 may also comprise an optical fiber or waveguide to transmit laser energy to the vagal nerve 17.

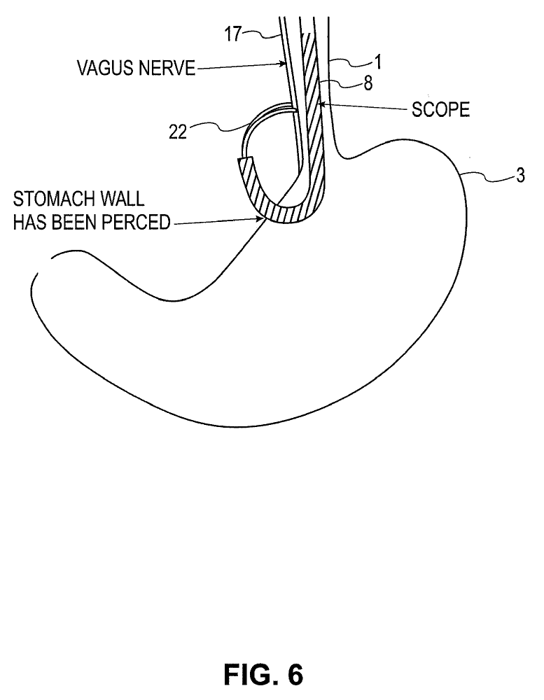

FIG. 6 illustrates another embodiment, in which a vagotomy is performed by mechanically disrupting the vagal nerve. In this embodiment, one or more medical devices 22 are advanced into the abdominal or thoracic cavity through the working channel of the endoscope 8, and positioned near the vagal nerve 17 to be disrupted. To mechanically disrupt the desired vagal nerve 17, the medical device 22 may dissect or ligate the vagal nerve. To do this, the medical device 22 may comprise a clip applier to ligate or crush the vagal nerve 17 with one or more clips on the nerve, or a cutting instrument, e.g., scissors, to dissect the vagal nerve 17.

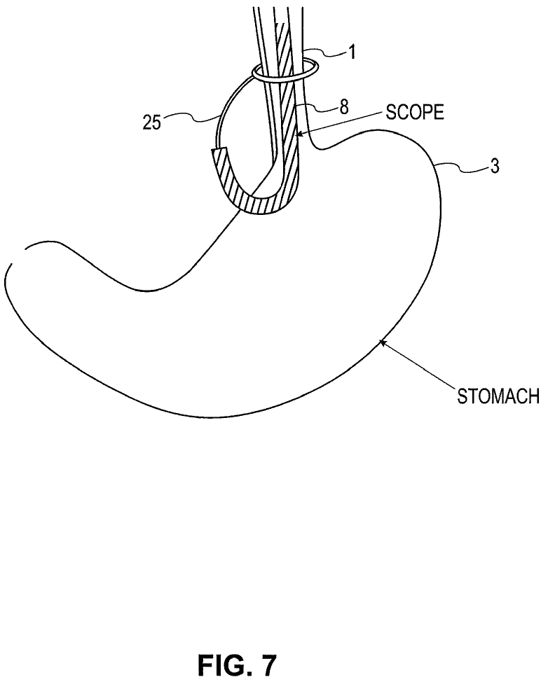

FIG. 7 illustrates yet another embodiment, in which gastric banding is performed to treat obesity by deploying a circular or other band 25 to mimic the effects of a laparoscopic band, in which the band is deployed around the upper portion of the stomach.

The vagotomy may be performed using any one of or a combination of the above methods. After the vagotomy is complete, the endoscope is withdrawn from the abdominal or thoracic cavity through the small opening. If the opening is small enough, the opening will seal itself. Alternatively a device may be used to seal the opening.

In the foregoing specification, the invention has been described with reference to specific embodiments thereof. It will, however, be evident that various modifications and changes may be made thereto without departing from the broader spirit and scope of the invention. As another example, each feature of one embodiment can be mixed and matched with other features shown in other embodiments. As yet another example, the order of steps of method embodiments may be changed. Features and processes known to those of ordinary skill may similarly be incorporated as desired. Additionally and obviously, features may be added or subtracted as desired. Accordingly, the invention is not to be restricted except in light of the attached claims and their equivalents.

1. A method for reducing obesity comprising:
positioning an endoscope within the esophagus or stomach;
advancing a needle through a working channel of the endoscope and extending the needle from the distal end of the endoscope;
inserting the needle into the esophagus or stomach to a position near a vagal nerve; and
delivering an agent from the needle to the vagal nerve, wherein the agent disrupts the vagal nerve.

2. The method of claim 1, further comprising:
inserting the needle into a space surrounding the vagal nerve; and
delivering the agent within the space surrounding the vagal nerve.

3. The method of claim 2, wherein the space is defined on one side by the esophagus and on mother side by the phreno-esophageal ligaments.

4. The method of claim 2, wherein the space is an enclosed space containing the delivered agent within the space.

5. The method of claim 2, further comprising suctioning the agent from the vagal nerve after a predetermined period of time.

6. The method of claim 1, further comprising:
inserting the needle into fat around the vagal nerve; and
delivering the agent within the fat around the vagal nerve.

7. The method of claim 1, wherein the agent comprises a hot liquid having a temperature sufficient to cause cellular death or damage.

8. The method of claim 1, wherein the agent comprises a cold liquid having a temperature sufficient to cause cellular death or damage.

9. The method of claim 1, wherein the agent comprises a toxin capable of causing cellular death or damage.

10. The method of claim 1, wherein the agent is capable of blocking the transmission of signals via the vagal nerve.

11. A method for reducing obesity comprising:
creating an opening through the esophagus or stomach;
advancing an endoscope within the esophagus or stomach through the opening;
advancing a medical device through a working channel of the endoscope and extending the medical device from the distal end of the endoscope;
positing the medical device near a vagal nerve on the outer surface of the esophagus or stomach; and
disrupting the vagal with the medical device.

12. The method of claim 11, wherein disrupting the vagal nerve comprises applying radio frequency (RF) energy to the vagal nerve from the medical device.

13. The method of claim 11, wherein disrupting the vagal nerve comprises applying ultrasound energy to the vagal nerve from the medical device.

14. The method of claim 11, wherein disrupting the vagal nerve comprises applying laser energy to the vagal nerve from the medical device.

15. The method of claim 11, wherein disrupting the vagal nerve comprises delivering an agent to the vagal nerve from the medical device, wherein the agent disrupts the vagal nerve.

16. The method of claim 15, wherein the agent comprises a hot or cold liquid having sufficient temperature to cause cellular death or damage.

17. The method of claim 11, wherein disrupting the vagal nerve comprises dissecting the vagal nerve with the medical device.

18. The method of claim 11, wherein disrupting the vagal nerve comprises ligating the vagal nerve with the medical device.

19. The method of claim 18, wherein ligating the vagal nerve comprises applying a clip on the vagal nerve to crush the vagal nerve.

20. The method of claim 11, wherein creating the opening comprises:
positioning the endoscope within the esophagus or stomach;
advancing a cutting instrument through a working channel of the endoscope and extending the cutting instrument from the distal end of the endoscope; and
piercing the opening through the esophagus or stomach with the cutting instrument.

21. The method of claim 11, further comprising retroflexing the distal portion of the endoscope upwards.
